(19) United States
(12) Patent Application Publication
(10) Pub. No.: US 2017/0291885 A1
(43) Pub. Date: Oct. 12, 2017

(54) METHOD FOR PREPARING ENAMIDE COMPOUND AND RUTHENIUM COMPLEX CATALYST USED THEREIN

(71) Applicant: POSTECH ACADEMY-INDUSTRY FOUNDATION, Pohang-si (KR)

(72) Inventors: Jai Wook PARK, Pohang-si (KR); Jin Yong PARK, Ulsan (KR); Yong Jin KIM, Busan (KR); Yea Rang KWON, Daegu (KR); Han Kyu PAK, Pohang-si (KR); Young Ho RHEE, Pohang-si (KR)

(21) Appl. No.: 15/473,756

(22) Filed: Mar. 30, 2017

(30) Foreign Application Priority Data
Apr. 7, 2016 (KR) 10-2016-0043015

(51) Int. Cl. C07D 333/20; C07D 307/52; C07C 319/12; B01J 31/22; C07D 317/58; C07D 213/40; C07D 311/68; C07C 231/10; C07C 253/30

(52) U.S. Cl. CPC C07D 333/20; C07C 231/10; C07D 307/52; C07C 319/12; C07C 253/30; C07D 317/58; C07D 213/40; C07D 311/68; B01J 31/2295; B01J 2531/821; B01J 2231/766

(57) ABSTRACT

Provided is a method for preparing an enamide compound, which includes reacting an organic azide compound having α-hydrogen and an anhydride by addition of a ruthenium complex catalyst in the presence of an ionic liquid, and a ruthenium complex catalyst used herein.

### CROSS-REFERENCE TO RELATED APPLICATION

This application claims priority to and the benefit of Korean Patent Application No. 10-2016-0043015, filed on 7 Apr. 2016, the disclosure of which is incorporated herein by reference in its entirety.

### BACKGROUND

### 1. Field of the Invention

The present invention relates to a method for preparing an enamide compound and a ruthenium complex catalyst used therein.

### 2. Discussion of Related Art

An enamide compound is a compound usefully used in carbon-carbon and carbon-nitrogen bond-forming reactions such as the aza-ene reaction, the Michael reaction, the Friedel-Craft reaction, cycloaddition and arylation. Particularly, as a precursor material for obtaining an optically pure amine compound, optically-selective hydrogenation is well known. Because of such a wide-range of applicability, conventionally, various methods for preparing an enamide compound have been studied.

FIG. 1 is a reaction scheme showing a variety of conventional methods for preparing an enamide compound.

As shown in FIG. 1, 1) acylation after the reaction between a nitrile and a Grignard reagent or methyl lithium, 2) the coupling reaction between a vinyl electrophile and an amide in the presence of a transition metal catalyst, 3) the Heck reaction between an aryl triflate or a halide and N-vinylacetamide, 4) the condensation between an amide and a ketone, 5) the reductive acylation of a ketoxime, and 6) the reaction between a ketone ammonia and an anhydride using a titanium complex are well known. However, the above-described reactions have limitations of a limited substrate range, difficulty in substrate preparation, low reactivity, limited functional group compatibility/tolerance, and a necessity of a specific additive.

### SUMMARY OF THE INVENTION

The present invention is directed to providing a method for preparing an enamide compound, which includes reacting an organic azide compound having α-hydrogen and an anhydride by addition of a ruthenium complex catalyst in the presence of an ionic liquid.

However, technical problems to be solved in the present invention are not limited to the above-described problems, and other problems that are not described herein will be clearly understood by those of ordinary skill in the art from the following descriptions.

The present invention provides a method for preparing an enamide compound, which includes reacting an organic azide compound having α-hydrogen, represented by Formula 1, and an anhydride represented by Formula 2, by addition of a ruthenium complex catalyst in the presence of an ionic liquid:

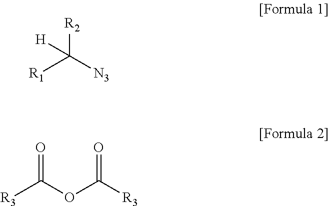

In Formula 1 or 2, R₁, R₂ and R₃ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group.

The reaction may be performed by addition of a ruthenium complex catalyst represented by Formula 3 or 4:

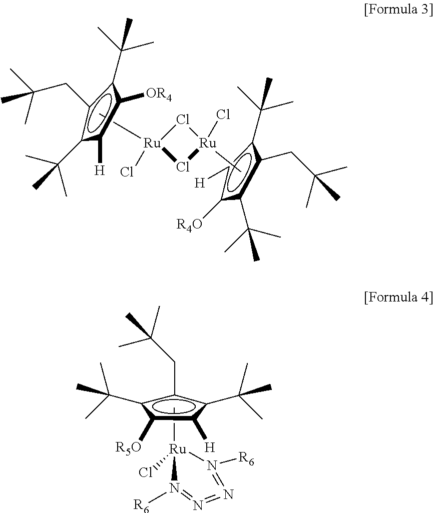

In Formula 3, R₄ is a substituted or unsubstituted C1 to C20 alkyl group, and in Formula 4, R₅ is a substituted or unsubstituted C1 to C20 alkyl group, and R₆ is a substituted or unsubstituted C1 to C20 alkyl group; a substituted or unsubstituted C6 to C20 aryl group; or a substituted or unsubstituted C3 to C20 heteroaryl group.

The ruthenium complex catalyst may be added at 1 to 5 mol % with respect to the organic azide compound having α-hydrogen.

The ruthenium complex catalyst and the anhydride may be added simultaneously.

The ruthenium complex catalyst may be added earlier than the anhydride to generate an imine intermediate, followed by addition of an anhydride.

The reaction may be performed in the presence of an ionic liquid represented by Formula 5:

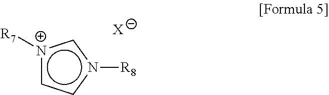

In Formula 5, R₇ and R₈ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group, and X is a halogen group.

The reaction may be performed at 50 to 120° C. for 30 minutes to 5 hours.

In one exemplary embodiment of the present invention, a ruthenium complex catalyst represented by Formula 3 used in the method for preparing an enamide compound is provided:

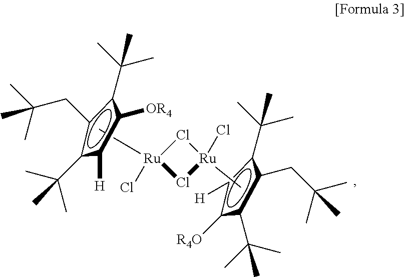

In Formula 3, R₄ is a substituted or unsubstituted C1 to C20 alkyl group.

The ruthenium complex catalyst may be prepared through the reaction of a compound represented by Formula 6 and RuCl₃(H₂O)₃:

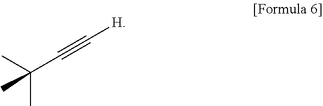

In another exemplary embodiment of the present invention, a ruthenium complex catalyst represented by Formula 4 used in the method for preparing an enamide compound is provided:

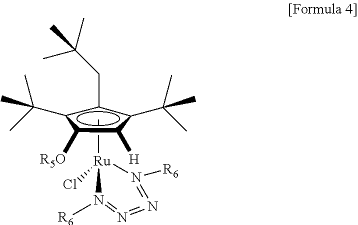

In Formula 4, R₅ is a substituted or unsubstituted C1 to C20 alkyl group, and R₆ is a substituted or unsubstituted C1 to C20 alkyl group; a substituted or unsubstituted C6 to C20 aryl group; or a substituted or unsubstituted C3 to C20 heteroaryl group.

The ruthenium complex catalyst may be prepared through the reaction of a compound represented by Formula 3 and a compound represented by Formula 1:

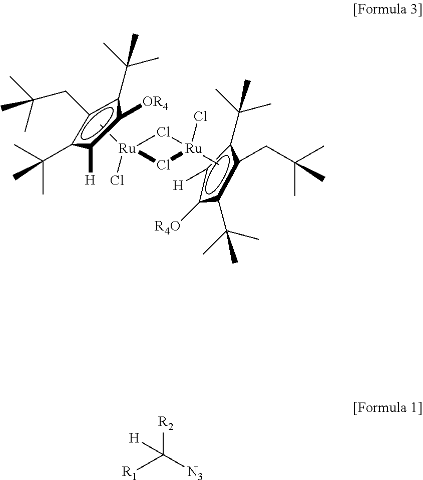

In Formula 3, R₄ is a substituted or unsubstituted C1 to C20 alkyl group, and in Formula 1, R₁ and R₂ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a reaction scheme showing a variety of conventional methods for preparing an enamide compound; and

FIG. 2 is a reaction scheme showing a method for preparing an enamide compound according to the present invention.

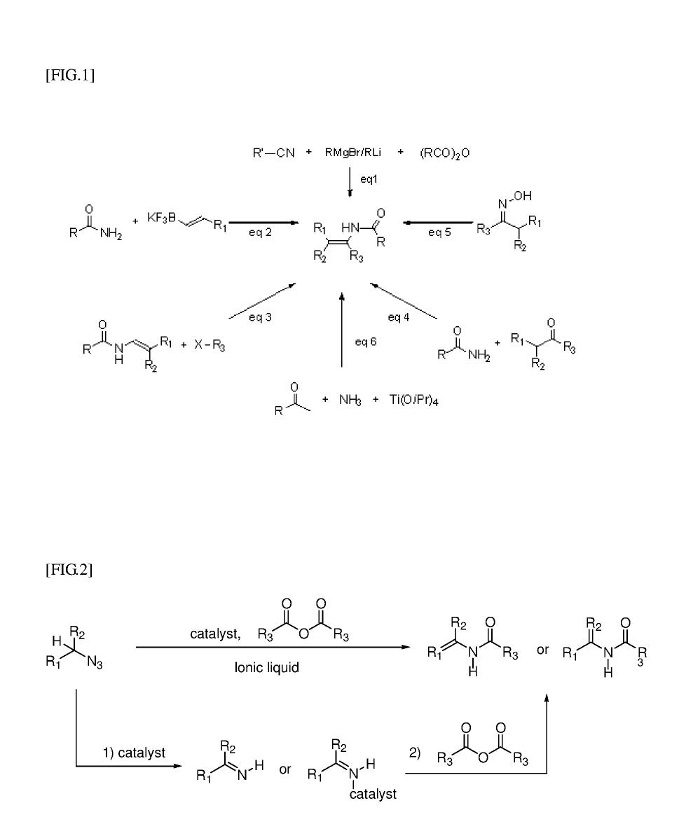

### DETAILED DESCRIPTION OF EXEMPLARY EMBODIMENTS

The present inventors identified that an enamide compound is able to be ideally prepared when an organic azide compound having α-hydrogen is reacted with an anhydride by addition of a specific ruthenium complex catalyst in the presence of an ionic liquid, and thus completed the present invention.

Hereinafter, the present invention will be described in detail.

The present invention provides a method for preparing an enamide compound, including reacting an organic azide compound having α-hydrogen, represented by Formula 1, and an anhydride represented by Formula 2, by addition of a ruthenium complex catalyst in the presence of an ionic liquid:

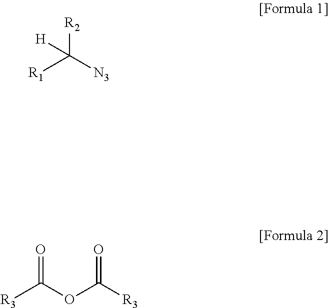

In Formula 1 or 2, R₁, R₂ and R₃ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group.

Specifically, the organic azide compound having α-hydrogen represented by Formula 1 refers to a compound having hydrogen at the a position of the azide compound, and may be, more specifically, 1-azidooctane, 1-azido-6-chlorohexane, 1-azido-6-(methoxymethoxy)hexane, ethyl-4-azidobutanoate, (2-azido-1-methoxyethyl)benzene, 1-azido-2-chlorooctane, (2-azido-1-chloroethyl)benzene, 3-(azidomethyl)pentane, (azidomethyl)cyclohexane, (2-azidoethyl)benzene, 1-(2-azidoethyl)-4-methoxybenzene, 1-(2-azidoethyl)-4-(trifluoromethyl)benzene, 2-(2-azidoethyl)furan, 2-(2-azidoethyl)thiophene, (2-azidoethyl)benzene, 1-(2-azidoethyl)-4-methoxybenzene, 1-(2-azidoethyl)-4-(trifluoromethyl)benzene, (3-azidopropyl)benzene, 2-azidooctane, (2-azidopropyl)benzene, 1-azido-2-methylcyclohexane, 1-azido-2-methylcyclopentane, (1-azidoethyl)benzene, 1-(1-azidoethyl)-2-methylbenzene, 1-(1-azidoethyl)-3-methylbenzene, 1-(1-azidoethyl)-4-methylbenzene, 1-(1-azidoethyl)-4-methoxybenzene, methyl-4-(1-azidoethyl)benzoate, 1-(1-azidoethyl)-4-nitrobenzene, 1-(1-azidoethyl)-4-chlorobenzene, 1-(1-azidoethyl)-4-bromobenzene, 1-(1-azidoethyl)-4-iodobenzene, (4-(1-azidoethyl)phenyl)(methyl)sulfane, N-(4-(1-azidoethyl)phenyl)acetamide, 4-(1-azidoethyl)benzonitrile, 1-(1-azidoethyl)-4-(chloromethyl)benzene, 5-(1-azidoethyl)benzo[d][1,3]dioxole, 2-(1-azidoethyl)naphthalene, 4-(1-azidoethyl)pyridine, 2-(1-azidoethyl)thiophene, 2-(1-azidoethyl)furan, (azido(cyclohexyl)methyl)benzene, (1-azidoethane-1,2-diyl)dibenzene, 4-azidochroman, 1-azido-2,3-dihydro-1H-indene, 1-azido-1,2,3,4-tetrahydronaphthalene, 5-azido-6,7,8,9-tetrahydro-5H-benzo[7]annulene, 4-(1-azidoethyl)benzaldehyde, or 1-(4-(1-azidoethyl)phenyl)ethanone.

Also, the anhydride represented by Formula 2 refers to an organic compound from which a water molecule is removed from two carboxyl groups, and more specifically, may be an acetic anhydride or a benzoic anhydride.

The organic azide compound having α-hydrogen, represented by Formula 1, and the anhydride represented by Formula 2 may be simultaneously reacted, or an intermediate of the organic azide compound having α-hydrogen, represented by Formula 1, may be reacted with the anhydride represented by Formula 2.

The term “ruthenium complex” used herein refers to a material containing a complex ion, which is formed by coordination of a ligand to ruthenium (atomic number 44), which is one of the transition metals, as a central metal ion. In addition, the term “ionic liquid” used herein refers to a liquid prepared by melting ion crystals.

Due to the addition of the ruthenium complex catalyst and the presence of the ionic liquid, the method according to the present invention can be performed in a one-step process for preparing an enamide compound, and is increased in reaction efficiency, an extended substrate spectrum, simplicity, practicability, and safety. Particularly, unlike a conventional method, the present method does not need severe reaction conditions and a specific additive.

FIG. 2 is a reaction scheme showing a method for preparing an enamide compound according to the present invention.

As shown in FIG. 2, the method for preparing an enamide compound, which includes reacting an organic azide compound having α-hydrogen and an anhydride by addition of a ruthenium complex catalyst in the presence of an ionic liquid, may be performed in a one-step process in which an enamide compound is finally produced through isomerization of N-acyl imine produced by simultaneously adding the ruthenium complex catalyst and the anhydride, or may be performed in a two-step process in which an enamide compound is finally produced by producing an imine (imine intermediate) having no substituent at nitrogen by adding the ruthenium complex catalyst, prior to the addition of the anhydride, to remove sequential nitrogens from the organic azide compound having α-hydrogen and performing 1,2-hydrogen rearrangement; and performing isomerization on N-acyl imine produced by reacting the imine (imine intermediate) with the anhydride.

The ruthenium complex catalyst may be represented by Formula 3 or Formula 4:

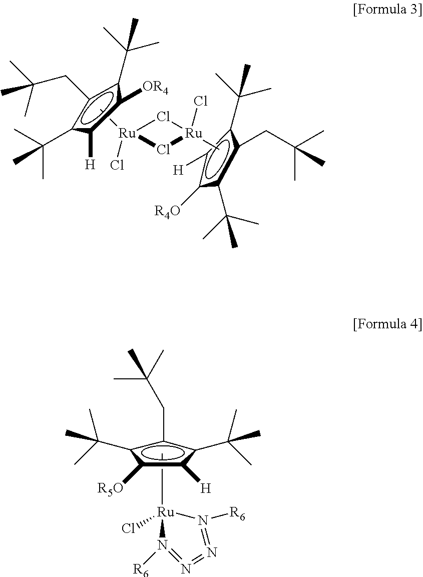

In Formula 3, R₄ is a substituted or unsubstituted C1 to C20 alkyl group, and in Formula 4, R₅ is a substituted or unsubstituted C1 to C20 alkyl group, and R₆ is a substituted or unsubstituted C1 to C20 alkyl group; a substituted or unsubstituted C6 to C20 aryl group; or a substituted or unsubstituted C3 to C20 heteroaryl group.

Specifically, in the ruthenium complex catalyst represented by Formula 3, R₄ is preferably hydrogen; or a substituted or unsubstituted C1 to C20 alkyl group, and may be selected in consideration of steric hindrance.

The ruthenium complex catalyst represented by Formula 3 may be prepared by reacting a compound represented by Formula 6 and RuCl₃(H₂O)₃:

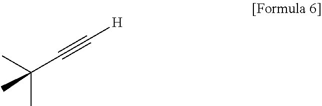

Here, the reaction for preparing the ruthenium complex catalyst may be performed in the presence of an organic solvent such as methanol or tetrahydrofuran at 50 to 55° C. for 12 to 48 hours.

Also, the ruthenium complex catalyst represented by Formula 4 may be prepared by reacting the compound represented by Formula 3 and the compound represented by Formula 1:

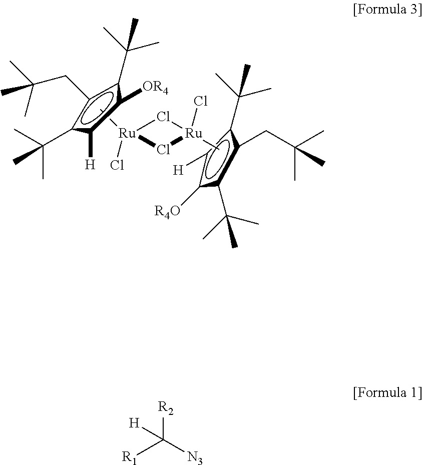

In Formula 3, R₄ is a substituted or unsubstituted C1 to C20 alkyl group, and in Formula 1, R₁ and R₂ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group.

Here, the reaction for preparing a ruthenium complex catalyst may be performed in the presence of an organic solvent such as methanol or tetrahydrofuran at 20 to 30° C. for 6 to 24 hours.

The ruthenium complex catalyst may be added at 1 to 5 mol %, and preferably 1 to 2 mol %, with respect to the organic azide compound having α-hydrogen, but the present invention is not limited thereto. Here, when the content of the ruthenium complex catalyst is very low, the effect caused by the catalyst is not properly exhibited, and when the content of the ruthenium complex catalyst is very high, economic feasibility is degraded.

The ruthenium complex catalyst may be added, prior to the addition of the anhydride, to remove sequential nitrogens from the organic azide compound having α-hydrogen, and 1,2-hydrogen rearrangement is performed, thereby producing an imine intermediate having no substituent at nitrogen, and the imine intermediate may react with the anhydride, leading to the two-step process. However, the one-step process performed by simultaneously adding the ruthenium complex catalyst and the anhydride is preferable in terms of process efficiency.

The ionic liquid may be represented by Formula 5:

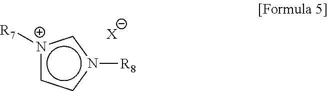

In Formula 5, R₇ and R₈ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group, and X is a halogen group.

Due to the presence of the ionic liquid as an environment-friendly liquid, the present method does not need severe reaction conditions and a specific additive, and has high safety.

Specifically, the ionic liquid represented by Formula 5 may be 1-octyl-3-methylimidazolium chloride or 1-butyl-3-methylimidazolium chloride.

The reaction may be performed at 50 to 120° C. for 30 minutes to 5 hours, but the present invention is not limited thereto. A smooth reaction may be sufficiently performed even under the above-described conditions of the reaction temperature and reaction time.

Also, the present invention provides a ruthenium complex catalyst represented by Formula 3 used in the method for preparing an enamide compound:

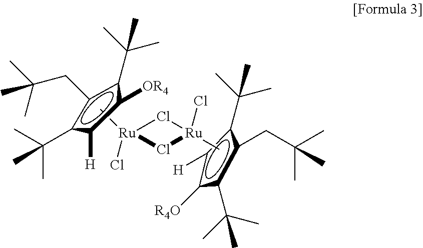

In Formula 3, R₄ is hydrogen; or a substituted or unsubstituted C1 to C20 alkyl group.

As described above, the ruthenium complex catalyst may be prepared by reacting the compound represented by Formula 6 and RuCl₃(H₂O)₃:

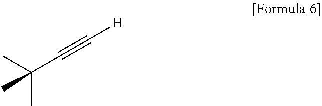

Also, the method provides a ruthenium complex catalyst represented by Formula 4 used in the method for preparing an enamide compound:

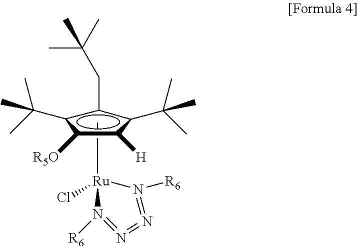

In Formula 4, R₅ is a substituted or unsubstituted C1 to C20 alkyl group, and R₆ is a substituted or unsubstituted C1 to C20 alkyl group; a substituted or unsubstituted C6 to C20 aryl group; or a substituted or unsubstituted C3 to C20 heteroaryl group.

As described above, the ruthenium complex catalyst may be prepared by reacting the compound represented by Formula 3 and the compound represented by Formula 1:

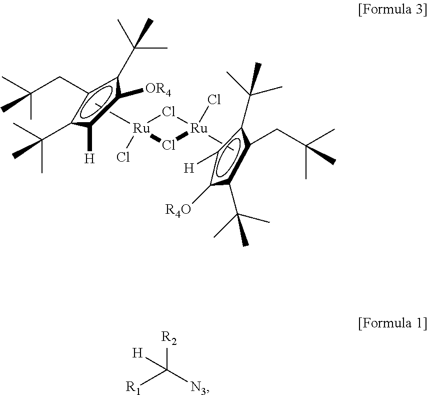

In Formula 3, R₄ is a substituted or unsubstituted C1 to C20 alkyl group, and in Formula 1, R₁ and R₂ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group.

Hereinafter, to help in understanding the present invention, exemplary examples will be provided. However, the following examples are merely provided to more fully understand the present invention, but the scope of the present invention is not limited by the following examples.

### Example 1-1: Preparation of Ruthenium Complex Catalyst (Formula 3)

A compound represented by Formula 6 and RuCl₃(H₂O)₃ (0.24 equivalents) were dissolved in methanol and reacted at 55° C. for 24 hours, thereby preparing a ruthenium complex catalyst represented by Formula 3:

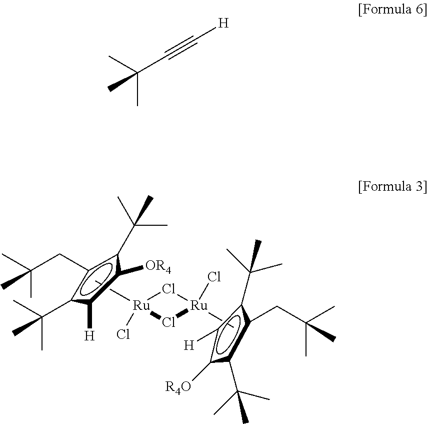

In Formula 3, R₄ is CH₃.

### Example 1-2: Preparation of Ruthenium Complex Catalyst (Formula 4)

A compound represented by Formula 3 and a compound represented by Formula 1 (8 equivalents) were dissolved in tetrahydrofuran and reacted at 25° C. for 12 hours, thereby preparing a ruthenium complex catalyst represented by Formula 4:

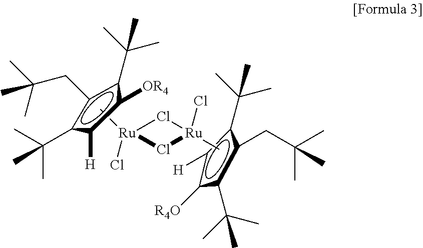

In Formula 3, R₄ is CH₃,

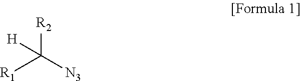

In Formula 1, R₁ is H, and R₂ is CH₂Ph, and

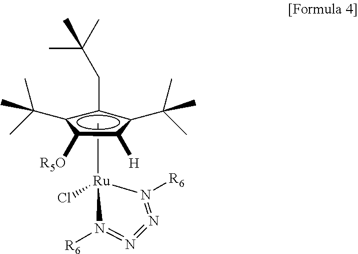

In Formula 4, R₅ is CH₃ and R₆ is CH₂Ph.

### Example 2-1: Preparation of Imine Intermediate by Addition of Ruthenium Complex Catalyst (Formula 3)

By addition of the ruthenium complex catalyst (Formula 3; 9.0 mg, 0.010 mmol) prepared in Example 1-1, 1-azidoethyl(benzene) (15 mg, 0.10 mmol) and triethylamine (2.0 mg, 0.020 mmol) were dissolved in 0.50 mL of tetrahydrofuran, and reacted under argon at 70° C. for 1 hour, thereby preparing an imine intermediate. Here, the imine intermediate having no substituent at a nitrogen atom was prepared in a yield of 99%.

### Example 2-2: Preparation of Imine Intermediate by Addition of Ruthenium Complex Catalyst (Formula 4)

By addition of a ruthenium complex catalyst (Formula 4; 15 mg, 0.022 mmol) prepared in Example 1-2, 1-azidoethyl(benzene) (16 mg, 0.11 mmol) was dissolved in 0.50 mL of tetrahydrofuran and reacted under argon at 70° C. for 3 hours, thereby preparing an imine intermediate. Here, the imine intermediate having no substituent at nitrogen was prepared in a yield of 99%.

### Example 3

By addition of the ruthenium complex catalyst (Formula 3; 9.0 mg, 0.010 mmol) prepared in Example 1-1, 1-azidooctane (39 mg, 0.25 mmol), triethylamine (50 mg, 0.50 mmol) and acetic anhydride (51 mg, 0.50 mmol) were dissolved in 1.0 mL of 1-octyl-3-methylimidazolium chloride and stirred under argon at 70° C. for 3 hours. After the reaction, the solvent was discarded, and a resulting precipitate was isolated, thereby preparing N-(oct-1-enyl)acetamide in a yield of 97%. Here, (E)- and (Z)-geometric isomers were identified in a ratio of 58:42.

### (E)-N-(oct-1-enyl)acetamide

¹H NMR (300 MHz, CDCl₃): δ=7.10 (bs, 1H), 6.72 (ddt, J=14.5, 10.4, 1.4 Hz, 1H), 5.12 (dt, J=14.3, 7.10, 1H), 1.89-2.08 (m, overlap, 2H), 2.03 (s, overlap, 3H), 1.33 (m, 8H), 0.876 (t, J=6.48 Hz, 3H); ¹³C NMR (75 MHz, CDCl₃): δ=167.8, 122.6, 113.7, 31.8, 30.0, 29.9, 28.9, 23.2, 22.8, 14.2; IR(KBr): ν=3298, 2926, 1657, 1520, 1275, 1267 cm⁻¹; HRMS (EI): calcd. for C₁₀H₁₉NO:169.1467. found:169.1467.

### (Z)-N-(oct-1-enyl)acetamide

¹H NMR (300 MHz, CDCl₃): δ=7.42 (bs, 1H), 6.67 (ddt, J=11.3, 9.1, 1.6 Hz, 1H), 4.71 (dt, J=8.8, 7.4 Hz, 1H), 2.08 (s, overlap, 3H), 2.01 (q, J=7.3 Hz, 2H), 1.20-1.46 (m, 8H), 0.886 (t, J=6.94 Hz, 3H); ¹³C NMR (75 MHz, CDCl₃): δ=167.7, 120.9, 111.6, 31.9, 29.5, 29.0, 25.9, 23.4, 22.8, 14.2; IR(KBr):ν=3276, 2926, 1652, 1538, 1261 cm⁻¹; HRMS (EI): calcd. for C₁₀H₁₉NO:169.1467. found:164.1467.

### Example 4

By addition of the ruthenium complex catalyst prepared in Example 1-1 (4.5 mg, 0.0050 mmol), 1-azido-6-chlorohexane (40 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(6-chlorohex-1-en-1-yl)acetamide in a yield of 81%. Here, (E)- and (Z)-geometrical isomers were identified in a ratio of 51:48.

### (E)-N-(6-chlorohex-1-en-1-yl)acetamide

¹H NMR (300 MHz, CDCl₃): δ=7.24 (bd, J=9.65 1H, 1H), 6.75 (ddt, J=14.1, 10.4, 1.3 Hz, 1H), 5.11 (dt, J=14.2, 7.1, 1H), 3.53 (t, J=6.6 Hz, 2H), 1.99-2.11 (m, overlap, 2H), 2.03 (s, overlap, 3H), 1.70-1.86 (m, 2H), 1.44-1.60 (m, 2H); ¹³C NMR (75 MHz, CDCl₃): δ=167.4, 123.2, 112.3, 45.1, 32.1, 29.1, 27.2, 23.4; IR(KBr):ν=3276, 2934, 1657, 1538, 1276 cm⁻¹ HRMS (EI): calcd. for C₈H₁₄ClNO:175.0764. found:175.0764.

### (Z)-N-(6-chlorohex-1-en-1-yl)acetamide

¹H NMR (300 MHz, CDCl₃): δ=7.28 (bs, 1H), 6.71 (ddt, J=11.4, 9.1, 1.5 Hz, 1H), 4.71 (dt, J=8.6, 7.4 Hz, 1H), 3.56 (t, J=6.51 Hz, 2H), 2.08 (s, overlap, 3H), 1.98-2.07 (m, overlap, 2H), 1.74-1.88 (m, 2H), 1.48-1.65 (m, 2H); ¹³C NMR (75 MHz, CDCl₃): δ=167.7, 121.6, 110.4, 45.1, 32.0, 26.7, 25.1, 23.6; IR(KBr): ν=3295, 1652, 1520, 1276, 1267 cm⁻¹ HRMS (EI): calcd. for C₈H₁₄ClNO:175.0764. found:175.0767.

### Example 5

By addition of the ruthenium complex catalyst prepared in Example 1-1 (4.5 mg, 0.0050 mmol), 1-azido-6-(methoxymethoxy)hexane (47 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(6-(methoxymethoxy)hex-1-en-1-yl)acetamide in a yield of 72%. Here, (E)- and (Z)-geometrical isomers were identified in a ratio of 55:45.

### (E)-N-(6-(methoxymethoxy)hex-1-en-1-yl)acetamide

¹H NMR (300 MHz, CDCl₃): δ=7.01 (bs, 1H), 6.74 (ddt, J=14.0, 10.6, 1.5 Hz, 1H), 5.11 (dt, J=14.0, 7.1 Hz, 1H), 4.61 (s, 3H), 3.52 (t, J=6.39 Hz, 2H), 3.36 (s, 3H), 2.05 (m, overlap, 2H), 2.02 (s, overlap, 3H), 1.64-1.42 (m, 4H); ¹³C NMR (75 MHz, CDCl₃): δ=167.2, 122.9, 112.7, 96.6, 67.8, 55.4, 29.6, 29.3, 26.7, 23.5; IR(KBr): ν=3279, 2931, 1657, 1538, 1275, 1261 cm⁻¹; HRMS (EI): calcd. for C₁₀H₁₉NO₃:201.1365. found: 201.1365.

### (Z)-N-(6-(methoxymethoxy)hex-1-en-1-yl)acetamide

¹H NMR (300 MHz, CDCl₃): δ=7.27 (bs, 1H), 6.72 (t, J=9.40 Hz, 1H), 4.72 (m, overlap, 1H), 4.64 (s, 2H), 3.57 (t, J=6.06, 2 H), 3.37 (s, 3H), 2.03-2.12 (m, overlap, 5H), 1.49-1.66 (m, 4H); ¹³C NMR (75 MHz, CDCl₃): δ=167.5, 121.6, 110.8, 69.7, 68.2, 55.5, 28.9, 26.6, 25.6, 23.6; IR(KBr):ν=3302, 2932, 1658, 1520, 1275, 1261 cm⁻¹; HRMS (EI): calcd. for C₁₀H₁₉NO₃:201.1365. found:201.1365.

### Example 6

By addition of the ruthenium complex catalyst prepared in Example 1-1 (4.5 mg, 0.0050 mmol), ethyl-4-azidobutanoate (39 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing ethyl-4-acetamidobut-3-enoate in a yield of 96%. Here, (E)- and (Z)-geometrical isomers were identified in a ratio of 34:66.

### (E)-ethyl-4-acetamidobut-3-enoate

¹H NMR (300 MHz, CDCl₃): δ=7.57 (bs, 1H), 6.86 (ddt, J=14.7, 10.6, 1.3, 1H), 5.24 (dt, J=14.5, 7.2 Hz, 1H), 4.14 (q, J=7.1 Hz, 2H), 3.05 (dd, J=7.3, 1.2 Hz, 2H), 2.04 (s, 2H), 1.27 (t, J=7.2 Hz, 3H); ¹³C NMR (75 MHz, CDCl₃): δ=167.0, 146.0, 134.5, 129.2, 128.1, 101.1, 56.3, 23.6; IR(KBr): ν=3306, 2985, 1652, 1520, 1275, 1261 cm⁻¹ HRMS (EI): calcd. for C₈H₁₃NO₃:171.0895. found:171.0895.

### (Z)-ethyl-4-acetamidobut-3-enoate

¹H NMR (300 MHz, CDCl₃): δ=8.24 (bs, 1H), 6.83 (dd, J=9.0, 10.9 Hz, 1H), 4.82 (dt, J=8.3, 7.7 Hz, 1H), 4.12 (q, J=7.1 Hz, 2H), 3.03 (dd, J=7.5, 0.8 Hz, 2H), 2.06 (s, 3H), 1.25 (t, J=7.1 Hz, 3H); ¹³C NMR (75 MHz, CDCl₃): δ=172.2, 168.2, 125.3, 101.3, 61.4, 32.2, 23.5, 14.3; IR(KBr): ν=3291, 2984, 1738, 1689, 1525, 1275, 1261 cm⁻¹ HRMS (EI): calcd. for C₈H₁₃NO₃:171.0895. found:171.0895.

### Example 7

By addition of the ruthenium complex catalyst prepared in Example 1-1 (4.5 mg, 0.0050 mmol), (2-azido-1-methoxyethyl)benzene (44 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(2-methoxy-2-phenylvinyl)acetamide in a yield of 77%. Here, (E)- and (Z)-geometrical isomers were identified in a ratio of 21:79.

### (E)-N-(2-methoxy-2-phenylvinyl)acetamide

¹H NMR (300 MHz, CDCl₃): δ=7.35-7.48 (m, 5H), 6.82 (bs, 1H), 6.53 (d, J=10.0 Hz, 1H), 3.69 (s, 3H), 1.99 (s, 3H); ¹³C NMR (75 MHz, CDCl₃): δ=167.0, 146.0, 134.5, 129.3, 129.2, 128.1, 101.1, 56.3, 23.6; IR(KBr): ν=3263, 1652, 1516, 1491, 1275, 1268 cm⁻¹ HRMS (EI): calcd. for C₁₁H₁₃NO₂:191.0946. found:191.0946.

### (Z)-N-(2-methoxy-2-phenylvinyl)acetamide

¹H NMR (300 MHz, CDCl₃): δ=7.65 (bs, 1H), 7.24-7.42 (m, 5H), 6.94 (d, J=10.5 Hz, 1H), 3.59 (s, 3H), 2.14 (s, 3H); ¹³C NMR (75 MHz, CDCl₃): δ=167.1, 142.3, 133.4, 128.8, 127.9, 125.4, 109.3, 58.5, 23.6; IR(KBr): ν=3231, 1648, 1513, 1490, 1274, 1263 cm⁻¹ HRMS (EI): calcd. for C₁₁H₁₃NO₂:191.0946. found:191.0945.

### Example 8

By addition of the ruthenium complex catalyst prepared in Example 1-1 (4.5 mg, 0.0050 mmol), 1-azido-2-chlorooctane (51 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(2-chlorooct-1-en-1-yl)acetamide in a yield of 81%. Here, (E)- and (Z)-geometrical isomers were identified in a ratio of 37:63.

### (E)-N-(2-chlorooct-1-en-1-yl)acetamide

¹H NMR (300 MHz, CDCl₃): δ=7.30 (bd, J=9.6 Hz, 1H), 6.93 (d, J=10.5 Hz, 1H), 2.29 (t, J=7.2 Hz, 2H), 2.06 (s, 3H), 1.50-1.57 (m, 2H), 1.22-1.33 (m, 6H), 0.88 (t, J=6.4 Hz, 3H); ¹³C NMR (75 MHz, CDCl₃): δ=167.3, 121.2, 120.2, 32.9, 31.8, 28.6, 27.0, 23.4, 22.8, 21.2, 14.3; IR(KBr): ν=3276, 2934, 1657, 1538, 1276, 1261 cm⁻¹ HRMS (EI): calcd. for C₁₀H₁₈ClNO:203.1077. found:203.1078.

### (Z)-N-(2-chlorooct-1-en-1-yl)acetamide

¹H NMR (300 MHz, CDCl₃): δ=7.28 (bs, 1H), 6.93 (dt, J=10.6, 0.9 Hz, 1H), 2.27 (td, J=7.4, 0.7 Hz, 2H), 2.08 (s, 3H), 1.43-1.58 (m, 2H), 1.20-1.33 (m, 6H), 0.86 (t, J=6.6 Hz, 3H); ¹³C NMR (75 MHz, CDCl₃): δ=167.3, 118.1, 117.8, 36.4, 31.7, 28.3, 27.4, 23.5, 22.7, 14.3; IR(KBr): ν=3295, 1652, 1520, 1371, 1276, 1267 cm⁻¹ HRMS (EI): calcd. for C₁₀H₁₈ClNO:203.1077. found:203.1074.

### Example 9

By addition of the ruthenium complex catalyst prepared in Example 1-1 (4.5 mg, 0.0050 mmol), (2-azido-1-chloroethyl)benzene (45 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(2-chloro-2-phenylvinyl)acetamide in a yield of 50%. All of the final products were identified as (E)-geometrical isomers.

¹H NMR (300 MHz, CDCl₃): δ=7.62 (bs, 2H), 7.54 (m, 2H), 7.23-7.41 (m, 3H), 2.18 (s, 3H); ¹³C NMR (75 MHz, CDCl₃): δ=167.5, 135.6, 128.7, 128.3, 125.7, 119.2, 115.7, 23.6; IR(KBr): ν=3276, 2934, 1657, 1538, 1276, 1261 cm⁻¹ HRMS (EI): calcd. for C₁₀H₁₀ClNO:195.0451. found:195.0450.

### Example 10

By addition of the ruthenium complex catalyst prepared in Example 1-1 (4.5 mg, 0.0050 mmol), 3-(azidomethyl)pentane (32 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(2-ethylbut-1-en-1-yl)acetamide in a yield of 99%.

¹H NMR (300 MHz, CDCl₃): δ=7.26 (bs, 1H), 6.46 (d, J=10.4 Hz, 1H), 1.96-2.07 (m. overlap, 7H), 0.97 (t, J=7.4 Hz. 3H), 0.96 (t, J=7.6 Hz. 3H); ¹³C NMR (75 MHz, CDCl₃): δ=167.5, 126.7, 115.8, 26.8, 23.4, 22.1, 12.9, 12.5; IR(KBr): ν=3297, 3197, 2965, 1652, 1267, 1212 cm⁻¹; HRMS (EI): calcd. for C₈H₁₅NO:141.1154. found:141.1152.

### Example 11

By addition of the ruthenium complex catalyst prepared in Example 1-1 (4.5 mg, 0.0050 mmol), (azidomethyl)cyclohexane (35 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(cyclohexylidenemethyl)acetamide in a yield of 94%.

¹H NMR (300 MHz, CDCl₃): δ=7.18 (bs, 1H), 6.46 (d, J=10.3 Hz, 1H), 2.00-2.10 (m, overlap, 4H), 2.03 (s, overlap, 3H), 1.42-1.60 (m, 6H); ¹³C NMR (75 MHz, CDCl₃): δ=167.6, 123.6, 114.1, 33.7, 28.3, 27.7, 27.2, 26.7, 23.5; IR(KBr): ν=3305, 3199, 2924, 1652, 1274, 1267 cm⁻¹; HRMS (EI): calcd. for C₉H₁₅NO:153.1154. found:153.1152.

### Example 12

By addition of the ruthenium complex catalyst prepared in Example 1-1 (4.5 mg, 0.0050 mmol), (2-azidoethyl)benzene (37 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-styrylacetamide in a yield of 98%. Here, (E)- and (Z)-geometrical isomers were identified in a ratio of 70:30.

### Example 13

By addition of the ruthenium complex catalyst prepared in Example 1-1 (4.5 mg, 0.0050 mmol), 1-(2-azidoethyl)-4-methoxybenzene (44 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(4-methoxystyryl)acetamide in a yield of 95%. Here, (E)- and (Z)-geometrical isomers were identified in a ratio of 68:32.

### Example 14

By addition of the ruthenium complex catalyst prepared in Example 1-1 (4.5 mg, 0.0050 mmol), 1-(2-azidoethyl)-4-(trifluoromethyl)benzene (54 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(4-(trifluoromethyl)styryl)acetamide in a yield of 98%. Here, (E)- and (Z)-geometrical isomers were identified in a ratio of 69:31.

### (Z)-N-(4-(trifluoromethyl)styryl)acetamide

¹H NMR (300 MHz, CDCl₃): δ=7.61-7.70 (m, 2H), 7.51 (bs, 1H), 7.35-7.42 (m, 2H), 0.05 (dd, J=12.0, 9.7 Hz, 1H), 5.75 (d, J=9.6 Hz, 1H), 2.09 (s, 3H); ¹³C NMR (75 MHz, CDCl₃): δ=167.9, 140.0, 128.9, 128.4, 126.3, 126.3, 123.9, 108.4, 23.8; ¹⁹F NMR (282 MHz, CDCl₃): δ=−62.6; IR(KBr): ν=3281, 1676, 1652, 1523, 1502, 1322, 1274 cm⁻¹; HRMS (EI): calcd. for C₁₁H₁₀F₃NO:229.0714. found:229.0711.

### Example 15

By addition of the ruthenium complex catalyst prepared in Example 1-1 (4.5 mg, 0.0050 mmol), 2-(2-azidoethyl)furan (38 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(2-(furan-2-yl)vinyl)acetamide in a yield of 92%. Here, (E)- and (Z)-geometrical isomers were identified in a ratio of 73:27.

### (E)-N-(2-(furan-2-yl)vinyl)acetamide

¹H NMR (300 MHz, CDCl₃): δ=7.74 (bs, 1H), 7.44 (dd, J=14.3, 10.9 Hz, 1H), 7.27-7.33 (m, 1H), 6.34 (dd, J=3.3, 1.9 Hz, 1H), 6.12 (d, J=3.2 Hz, 1H), 5.99 (d, J=14.5 Hz, 1H), 2.11 (s, 3H); ¹³C NMR (75 MHz, CDCl₃): δ=167.8, 151.7, 141.5, 122.3, 111.5, 106.3, 102.2, 23.6; IR(KBr): ν=3244, 1672, 1651, 1268, 1261 cm⁻¹; HRMS (EI): calcd. for C₈H₉NO₂:151.0633. found:151.0633.

### (Z)-N-(2-(furan-2-yl)vinyl)acetamide

¹H NMR (300 MHz, CDCl₃): δ=8.77 (bs, 1H), 7.46 (d, J=1.1 Hz, 1H), 6.87 (dd, J=12.2, 9.7 Hz, 1H), 6.36-6.48 (m, 1H), 6.18 (d, J=3.3 Hz, 1H), 5.49 (d, J=9.6 Hz, 1H), 2.17 (s, 3H); ¹³C NMR (75 MHz, CDCl₃): δ=167.6, 153.01, 141.5, 120.2, 111.7, 108.1, 97.7, 23.9; IR(KBr): ν=3382, 2921, 1734, 1652, 1275, 1261 cm⁻¹; HRMS (EI): calcd. for C₈H₉NO₂:151.0633. found:151.0633.

### Example 16

By addition of the ruthenium complex catalyst prepared in Example 1-1 (4.5 mg, 0.0050 mmol), 2-(2-azidoethyl)thiophene (38 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(2-(thiophen-2-yl)vinyl)acetamide in a yield of 93%. Here, (E)- and (Z)-geometrical isomers were identified in a ratio of 64:36.

### (E)-N-(2-(thiophen-2-yl)vinyl)acetamide

¹H NMR (300 MHz, CDCl₃): δ=7.80 (bs, 1H), 7.37 (dd, J=14.7, 10.7 Hz, 1H), 7.07 (d, J=5.0. Hz, 1H), 6.97 (dd, J=5.2, 3.5 Hz, 1H), 6.86 (d, J=3.5 Hz, 1H), 6.29 (d, J=14.5 Hz, 1H), 2.11 (s, 3H); ¹³C NMR (75 MHz, CDCl₃): δ=167.8, 140.6, 127.6, 124.5, 123.2, 122.6, 28, 106.9, 23.5; IR(KBr): ν=3270, 1670, 1643, 1542, 1275, 1261 cm⁻¹; HRMS (EI): calcd. for C₈H₉NOS:167.0405. found:167.0405.

### (Z)-N-(2-(thiophen-2-yl)vinyl)acetamide

¹H NMR (300 MHz, CDCl₃): δ=7.71 (bs, 1H), 7.27 (d, J=4.8 Hz, 1H), 7.05 (dd, J=5.2, 3.6 Hz, 1H), 6.97 (d, J=3.5 Hz, 1H), 6.90 (dd, J=12.1, 9.8 Hz, 1H), 5.89 (d, J=9.6 Hz, 1H), 2.14 (s, 3H); ¹³C NMR (75 MHz, CDCl₃): δ=167.7, 138.4, 127.8, 126.2, 124.4, 121.2, 103.1, 23.8; IR(KBr): ν=3286, 1677, 1645, 1523, 1489, 1276, 1260 cm⁻¹; HRMS (EI): calcd. for C₈H₉NOS:167.0405. found:167.0405.

### Example 17

By addition of the ruthenium complex catalyst prepared in Example 1-1 (4.5 mg, 0.0050 mmol), (2-azidoethyl)benzene (37 mg, 0.25 mmol), triethylamine (50 mg, 0.50 mmol) and benzoic anhydride (110 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-styrylbenzamide in a yield of 99%. All of the final products were identified as (E)-geometrical isomers.

### (E)-N-styrylbenzamide

¹H NMR (300 MHz, DMSO-d₆): δ=10.7 (bd, J=9.8 Hz, 1H), 8.00 (m, 2H), 7.68 (dd, J=14.8, 9.9 Hz, 1H), 7.48-7.63 (m, 3H), 7.36-7.45 (m, 2H), 7.26-7.35 (m, 2H), 7.11-7.23 (m, 1H), 6.49 (d, J=14.7 Hz, 1H); ¹³C NMR (75 MHz, DMSO-d₆): δ=164.1, 136.6, 133.4, 131.9, 128.8, 128.5, 127.7, 126.3, 125.3, 124.2, 113.0.

### Example 18

By addition of the ruthenium complex catalyst prepared in Example 1-1 (4.5 mg, 0.0050 mmol), 1-(2-azidoethyl)-4-methoxybenzene (37 mg, 0.25 mmol), triethylamine (50 mg, 0.50 mmol) and benzoic anhydride (110 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(4-methoxystyryl)benzamide in a yield of 99%. All of the final products were identified as (E)-geometrical isomers.

### (E)-N-(4-methoxystyryl)benzamide

¹H NMR (300 MHz, DMSO-d₆): δ=10.6 (bd, J=9.8 Hz, 1H), 7.89-8.05 (m, 2H), 7.46-7.64 (m, 4H), 7.27-7.39 (m, 2H), 6.83-6.95 (m, 2H), 6.43 (d, J=14.7 Hz, 1H), 3.74 (s, 3H); ¹³C NMR (75 MHz, DMSO-d₆): δ=163.9, 158.0, 133.5, 131.8, 129.0, 128.5, 127.6, 126.5, 122.4, 114.2, 112.8, 55.1.

### Example 19

By addition of the ruthenium complex catalyst prepared in Example 1-1 (4.5 mg, 0.0050 mmol), 1-(2-azidoethyl)-4-(trifluoromethyl)benzene (37 mg, 0.25 mmol), triethylamine (50 mg, 0.50 mmol) and benzoic anhydride (110 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(4-(trifluoromethyl)styryl)benzamide in a yield of 99%. All of the final products were identified as (E)-geometrical isomers.

### (E)-N-(4-(trifluoromethyl)styryl)benzamide

¹H NMR (300 MHz, DMSO-d₆): δ=10.8 (bd, J=10 Hz, 1H), 7.93-8.05 (m, 2H), 7.83 (dd, J=14.4, 10.0 Hz, 1H), 7.48-7.68 (m, 7H), 6.53 (d, J=14.7 Hz, 1H); ¹³C NMR (75 MHz, DMSO-d₆): δ=164.3, 141.1, 133.1, 132.1, 128.6, 127.8, 126.8, 126.0, 125.7, 125.6, 125.5, 111.3; IR(KBr): ν=3425, 1639, 1580, 1526, 1275, 1276 cm⁻¹; HRMS (EI): calcd. for C₁₆H₁₂F₃NO:291.0871. found:291.0873.

### Example 20

By addition of the ruthenium complex catalyst prepared in Example 1-1 (4.5 mg, 0.0050 mmol), (3-azidopropyl)benzene (40 mg, 0.25 mmol), triethylamine (50 mg, 0.50 mmol) and benzoic anhydride (110 mg, 0.50 mmol) were dissolved in 1.0 mL of 1-octyl-3-methylimidazolium chloride, and stirred under argon at 70° C. for 3 hours. After the reaction, the solvent was discarded, and the resulting precipitate was isolated, thereby preparing N-(3-phenylprop-1-en-1-yl)benzamide in a yield of 99%. Here, (E)- and (Z)-geometrical isomers were identified in a ratio of 34:66.

### (Z)-N-(3-phenylprop-1-en-1-yl)benzamide

¹H NMR (300 MHz, CDCl₃): δ=7.71 (bs, 1H), 7.16-7.56 (m, 10H), 7.02 (ddt, J=11.2, 9.0, 1.4 Hz, 1H), 5.09 (dt, J=8.9, 7.0 Hz, 1H), 3.49 (d, J=6.9 Hz, 2H). ¹³C NMR (75 MHz, CDCl₃): δ=164.4, 139.7, 133.7, 132.1, 129.1, 128.7, 128.6, 127.2, 126.8, 122.9, 109.7, 32.6; IR(KBr): ν=3330, 3062, 3029, 1726, 1666, 1511, 1276, 1261 cm⁻¹ HRMS (EI): calcd. for C₁₆H₁₅NO:237.1154. found:237.1154.

### Example 21

By addition of the ruthenium complex catalyst prepared in Example 1-1 (4.5 mg, 0.0050 mmol), 2-azidooctane (39 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 12 hours. After the reaction, the solvent was discarded, and the resulting precipitate was isolated, thereby preparing N-(oct-1-en-2-yl)acetamide in a yield of 23%, and N-(oct-2-en-2-yl)acetamide, which is a structural isomer, in a yield of 72%. Here, (E)- and (Z)-geometrical isomers were identified in a ratio of 90:10.

### Example 22

By addition of the ruthenium complex catalyst prepared in Example 1-1 (4.5 mg, 0.0050 mmol), (2-azidopropyl)benzene (39 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 12 hours. After the reaction, the solvent was discarded, and the resulting precipitate was isolated, thereby preparing N-(3-phenylprop-1-en-2-yl)acetamide in a yield of 13%, and N-(1-phenylprop-1-en-2-yl)acetamide, which is a structural isomer, in a yield of 47%. Here, (E)- and (Z)-geometrical isomers were identified in a ratio of 53:47.

### Example 23

By addition of the ruthenium complex catalyst prepared in Example 1-1 (4.5 mg, 0.0050 mmol), 1-azido-2-methylcyclohexane (35 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 12 hours. After the reaction, the solvent was discarded, and the resulting precipitate was isolated, thereby preparing N-(2-methylcyclohex-1-en-1-yl)acetamide in a yield of 53%, and N-(6-methylcyclohex-1-en-1-yl)acetamide, which is a structural isomer, in a yield of 47%.

### N-(6-methylcyclohex-1-en-1-yl)acetamide

¹H NMR (300 MHz, CDCl₃): δ=6.36 (bs, 1H), 6.05 (t, J=3.7 Hz, 1H), 2.23-2.43 (m, 1H), 2.06-2.16 (m, 2H), 2.04 (s, 3H), 1.73-1.87 (m, 1H), 1.38-1.70 (m, 3H), 1.07 (d, J=7.0 Hz, 3H); ¹³C NMR (75 MHz, CDCl₃): δ=168.8, 136.7, 115.2, 31.5, 30.8, 24.7, 24.6, 19.5, 19.0; ν=3286, 3197, 2931, 1652, 1579, 1261 cm⁻¹; HRMS (EI): calcd. for C₉H₁₅NO:153.1154. found:153.1154.

### Example 24

By addition of the ruthenium complex catalyst prepared in Example 1-1 (4.5 mg, 0.0050 mmol), 1-azido-2-methylcyclopentane (31 mg, 0.25 mmol), triethylamine (50 mg, 0.50 mmol) and acetic anhydride (51 mg, 0.50 mmol) were dissolved in 1.0 mL of 1-octyl-3-methylimidazolium chloride, and stirred under argon at 70° C. for 12 hours. After the reaction, the solvent was discarded, and the resulting precipitate was isolated, thereby preparing N-(2-methylcyclopent-1-en-1-yl)acetamide in a yield of 69%, and N-(5-methylcyclopent-1-en-1-yl)acetamide, which is a structural isomer, in a yield of 29%.

### N-(2-methylcyclopent-1-en-1-yl)acetamide

¹H NMR (300 MHz, CDCl₃): δ=6.81 (bs, 1H), 2.55-2.87 (m, 2H), 2.16-2.33 (m, 2H), 2.05 (s, 3H), 1.76-1.92 (m, 2H), 1.60 (s, 3H); ¹³C NMR (75 MHz, CDCl₃): δ=168.3, 131.0, 124.0, 35.5, 33.5, 23.9, 21.0, 13.3; IR(KBr): ν=3035, 2959, 1690, 1645, 1267 cm⁻¹; HRMS (EI): calcd. for C₈H₁₃NO:139.0997. found:139.0997.

### N-(5-methylcyclopent-1-en-1-yl)acetamide

¹H NMR (300 MHz, CDCl₃): δ=6.66 (bs, 1H), 5.98 (d, J=1.4 Hz, 1H), 2.61-2.85 (m, 1H), 2.23-2.53 (m, 2H), 1.94-2.20 (m, 4H), 1.34-1.52 (m, 1H), 1.08 (d, J=6.9 Hz, 3H); ¹³C NMR (75 MHz, CDCl₃): δ=168.5, 139.6, 111.4, 40.0, 30.7, 29.6, 24.4, 19.2; IR(KBr): ν=3289, 2957, 1671, 1549, 1268 cm⁻¹; HRMS (EI): calcd. for C₈H₁₃NO:139.0997. found:139.0997.

### Example 25

By addition of the ruthenium complex catalyst prepared in Example 1-1 (6.3 mg, 0.0070 mmol), (1-azidoethyl)benzene (1.0 g, 7.0 mmol), triethylamine (2.8 mg, 0.014 mmol) and acetic anhydride (890 mg, 8.4 mmol) were dissolved in 5.0 mL of 1-butyl-3-methylimidazolium chloride, and stirred under argon at 70° C. for 24 hours. After the reaction, the solvent was discarded, and the resulting precipitate was isolated, thereby preparing N-(1-phenylvinyl)acetamide in a yield of 91%.

### Example 26

By addition of the ruthenium complex catalyst prepared in Example 1-1 (2.2 mg, 0.0025 mmol), 1-(1-azidoethyl)-2-methylbenzene (40 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-o-tolylvinyl)acetamide in a yield of 78%.

### Example 27

By addition of the ruthenium complex catalyst prepared in Example 1-1 (2.2 mg, 0.0025 mmol), 1-(1-azidoethyl)-3-methylbenzene (40 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-m-tolylvinyl)acetamide in a yield of 99%.

### Example 28

By addition of the ruthenium complex catalyst prepared in Example 1-1 (2.2 mg, 0.0025 mmol), 1-(1-azidoethyl)-4-methylbenzene (40 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-p-tolylvinyl)acetamide in a yield of 99%.

### Example 29

By addition of the ruthenium complex catalyst prepared in Example 1-1 (2.2 mg, 0.0025 mmol), 1-(1-azidoethyl)-4-methoxybenzene (44 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-methoxyphenyl)vinyl)acetamide in a yield of 99%.

### Example 30

By addition of the ruthenium complex catalyst prepared in Example 1-1 (2.2 mg, 0.0025 mmol), methyl-4-(1-azidoethyl)benzoate (51 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing methyl-4-(1-cetamidovinyl)benzoate in a yield of 99%.

### Example 31

By addition of the ruthenium complex catalyst prepared in Example 1-1 (2.2 mg, 0.0025 mmol), 1-(1-azidoethyl)-4-nitrobenzene (48 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-nitrophenyl)vinyl)acetamide in a yield of 75%.

### Example 32

By addition of the ruthenium complex catalyst prepared in Example 1-1 (2.2 mg, 0.0025 mmol), 1-(1-azidoethyl)-4-chlorobenzene (45 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-chlorophenyl)vinyl)acetamide in a yield of 94%.

### Example 33

By addition of the ruthenium complex catalyst prepared in Example 1-1 (2.2 mg, 0.0025 mmol), 1-(1-azidoethyl)-4-bromobenzene (57 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-bromophenyl)vinyl)acetamide in a yield of 85%.

### Example 34

By addition of the ruthenium complex catalyst prepared in Example 1-1 (2.2 mg, 0.0025 mmol), 1-(1-azidoethyl)-4-iodobenzene (68 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-iodophenyl)vinyl)acetamide in a yield of 84%.

### Example 35

By addition of the ruthenium complex catalyst prepared in Example 1-1 (2.2 mg, 0.0025 mmol), (4-(1-azidoethyl)phenyl)(methyl)sulfane (48 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-(methylthio)phenyl)vinyl)acetamide in a yield of 71%.

### Example 36

By addition of the ruthenium complex catalyst prepared in Example 1-1 (2.2 mg, 0.0025 mmol), N-(4-(1-azidoethyl)phenyl)acetamide (51 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-acetamidophenyl)vinyl)acetamide in a yield of 40%.

### Example 37

By addition of the ruthenium complex catalyst prepared in Example 1-1 (2.2 mg, 0.0025 mmol), 4-(1-azidoethyl)benzonitrile (43 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-cyanophenyl)vinyl)acetamide in a yield of 82%.

### Example 38

By addition of the ruthenium complex catalyst prepared in Example 1-1 (2.2 mg, 0.0025 mmol), 1-(1-azidoethyl)-4-(chloromethyl)benzene (49 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-(chloromethyl)phenyl)vinyl)acetamide in a yield of 99%.

### N-(1-(4-(chloromethyl)phenyl)vinyl)acetamide

¹H NMR (300 MHz, CDCl₃): δ=7.30-7.50 (m, 4H), 7.06 (bs, 1H), 5.80 (s, 1H), 5.09 (s, 1H), 4.58 (s, 2H), 2.08 (s, 3H); ¹³C NMR (75 MHz, CD₃CN): δ=169.4, 140.2, 138.6, 138.1, 129.1, 126.7, 103.5, 45.9, 24.6; IR(KBr): ν=3254, 3156, 3032, 1667, 1629, 1550, 1275, 1262 cm⁻¹; HRMS (EI): calcd. for C₁₁H₁₂ClNO:209.0607. found:209.0607.

### Example 39

By addition of the ruthenium complex catalyst prepared in Example 1-1 (2.2 mg, 0.0025 mmol), 5-(1-azidoethyl)benzo[d][1,3]dioxole (48 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(benzo[d][1,3]dioxol-5-yl)vinyl)acetamide in a yield of 90%.

### Example 40

By addition of the ruthenium complex catalyst prepared in Example 1-1 (2.2 mg, 0.0025 mmol), 2-(1-azidoethyl)naphthalene (49 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(naphthalen-2-yl)vinyl)acetamide in a yield of 99%.

### Example 41

By addition of the ruthenium complex catalyst prepared in Example 1-1 (2.2 mg, 0.0025 mmol), 4-(1-azidoethyl)pyridine (37 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(pyridin-4-yl)vinyl)acetamide in a yield of 15%.

### Example 42

By addition of the ruthenium complex catalyst prepared in Example 1-1 (2.2 mg, 0.0025 mmol), 2-(1-azidoethyl)thiophene (38 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(thiophen-2-yl)vinyl)acetamide in a yield of 84%.

### Example 43

By addition of the ruthenium complex catalyst prepared in Example 1-1 (2.2 mg, 0.0025 mmol), 2-(1-azidoethyl)furan (34 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(furan-2-yl)vinyl)acetamide in a yield of 79%.

### Example 44

By addition of the ruthenium complex catalyst prepared in Example 1-1 (2.2 mg, 0.0025 mmol), (azido(cyclohexyl)methyl)benzene (54 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(cyclohexylidene(phenyl)methyl)acetamide in a yield of 99%.

### Example 45

By addition of the ruthenium complex catalyst prepared in Example 1-1 (2.2 mg, 0.0025 mmol), (1-azidoethane-1,2-diyl)dibenzene (56 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1,2-diphenylvinyl)acetamide in a yield of 97%. Here, (E)- and (Z)-geometrical isomers were identified in a ratio of 25:72.

### Example 46

By addition of the ruthenium complex catalyst prepared in Example 1-1 (2.2 mg, 0.0025 mmol), 4-azidochroman (44 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(2H-chromen-4-yl)acetamide in a yield of 85%.

### Example 47

By addition of the ruthenium complex catalyst prepared in Example 1-1 (2.2 mg, 0.0025 mmol), 1-azido-2,3-dihydro-1H-indene (40 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1H-inden-3-yl)acetamide in a yield of 97%.

### Example 48

By addition of the ruthenium complex catalyst prepared in Example 1-1 (2.2 mg, 0.0025 mmol), 1-azido-1,2,3,4-tetrahydronaphthalene (43 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(3,4-dihydronaphthalen-1-yl)acetamide in a yield of 95%.

### Example 49

By addition of the ruthenium complex catalyst prepared in Example 1-1 (2.2 mg, 0.0025 mmol), 5-azido-6,7,8,9-tetrahydro-5H-benzo[7]annulene (47 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(6,7-dihydro-5H-benzo[7]annulen-9-yl)acetamide in a yield of 60%.

### Example 50

By addition of the ruthenium complex catalyst prepared in Example 1-1 (2.2 mg, 0.0025 mmol), 4-(1-azidoethyl)benzaldehyde (44 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-formylphenyl)vinyl)acetamide in a yield of 51%.

### Example 51

By addition of the ruthenium complex catalyst prepared in Example 1-1 (2.2 mg, 0.0025 mmol), 1-(4-(1-azidoethyl)phenyl)ethanone (47 mg, 0.25 mmol), triethylamine (1.0 mg, 0.0050 mmol) and acetic anhydride (31 mg, 0.30 mmol) were dissolved in 1.0 mL of 1-butyl-3-methylimidazolium chloride, and stirred under argon at 70° C. for 3 hours. After the reaction, the solvent was discarded, and the resulting precipitate was isolated, thereby preparing N-(1-(4-acetylphenyl)vinyl)acetamide in a yield of 99%.

In Examples 3 to 51, the organic azide compound having α-hydrogen, which is a starting material, the enamide compound, which is a product, and its yield and a geometrical isomer ratio are shown in Table 1.

TABLE 1

| | | | Yield | Geometrical isomer |
|---|---|---|---|---|
| Example | Azide | Product | (%) | (E/Z) |
| 3 | 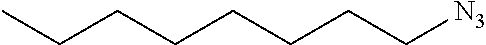 | 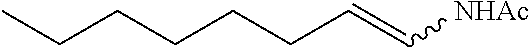 | 97 | 58:42 |
| 4 | 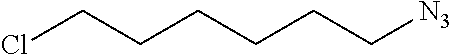 | 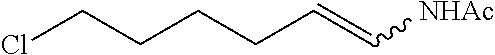 [SMILES: CC(=O)NC=CCCCCCl] | 81 | 52:48 |
| 5 | 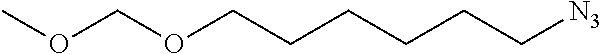 | 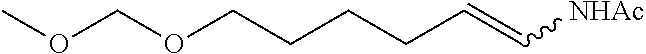 | 72 | 55:45 |
| 6 | 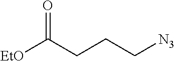 [SMILES: CCOC(=O)CCCN=[N+]=[N-]] | 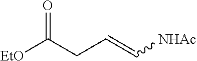 [SMILES: CCOC(=O)CC=CNC(C)=O] | 96 | 34:66 |
| 7 | 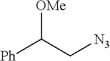 [SMILES: COC(CN=[N+]=[N-])c1ccccc1] | 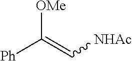 [SMILES: COC(=CNC(C)=O)c1ccccc1] | 77 | 21:79 |
| 8 | 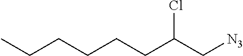 | 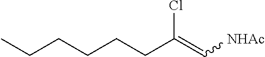 | 81 | 37:63 |
| 9 | 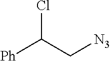 [SMILES: [N-]=[N+]=NCC(Cl)c1ccccc1] | 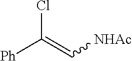 [SMILES: CC(=O)NC=C(Cl)c1ccccc1] | 50 | 100:0 |
| 10 | 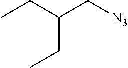 | 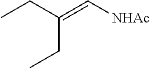 | 99 | |
| 11 | 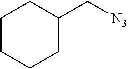 | 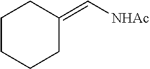 [SMILES: CC(=O)NC=C1CCCCC1] | 94 | |
| 12 | 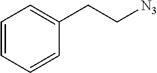 [SMILES: [N-]=[N+]=NCCc1ccccc1] | 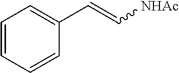 [SMILES: CC(=O)NC=Cc1ccccc1] | 98 | 70:30 |
| 13 | 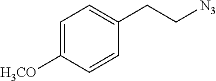 [SMILES: COc1ccc(CCN=[N+]=[N-])cc1] | 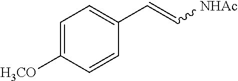 [SMILES: COc1ccc(C=CNC(C)=O)cc1] | 98 | 68:32 |
| 14 | 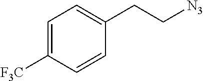 [SMILES: [N-]=[N+]=NCCc1ccc(C(F)(F)F)cc1] | 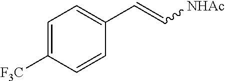 [SMILES: CC(=O)NC=Cc1ccc(C(F)(F)F)cc1] | 98 | 69:31 |
| 15 | 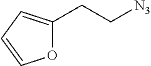 | 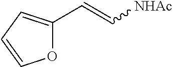 [SMILES: CC(=O)NC=Cc1ccco1] | 92 | 73:27 |
| 16 | 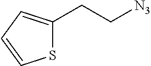 | 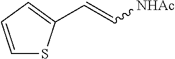 [SMILES: CC(=O)NC=Cc1cccs1] | 93 | 64:36 |
| 17 | 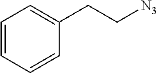 [SMILES: [N-]=[N+]=NCCc1ccccc1] | 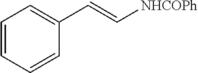 | 99 | |
| 18 | 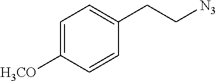 [SMILES: COc1ccc(CCN=[N+]=[N-])cc1] | 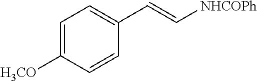 | 99 | |
| 19 | 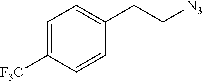 [SMILES: [N-]=[N+]=NCCc1ccc(C(F)(F)F)cc1] | 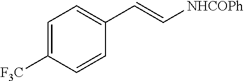 | 99 | |
| 20 | 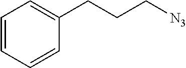 [SMILES: [N-]=[N+]=NCCCc1ccccc1] | 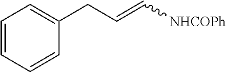 | 99 | 34:66 |
| 21 | 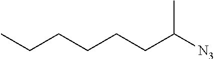 | 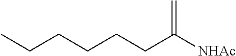 | 23 | |
| | | 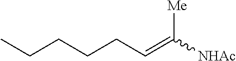 | 72 | 90:10 |
| 22 | 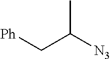 | 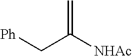 | 13 | |
| | | 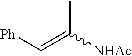 | 47 | 53:47 |
| 23 | 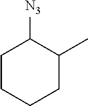 | 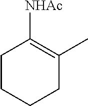 | 53 | |
| | | 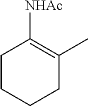 | 47 | |
| 24 | 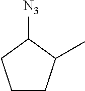 | 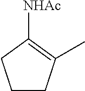 | 69 | |
| | | 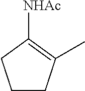 | 29 | |
| 25 | 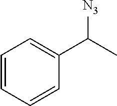 | 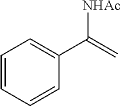 | 91 | |
| 26 | 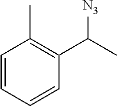 | 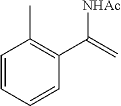 | 78 | |
| 27 | 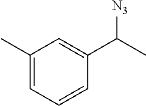 | 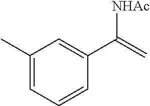 | 99 | |
| 28 | 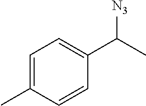 | 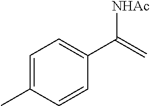 | 99 | |
| 29 | 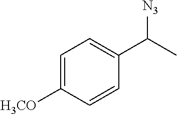 | 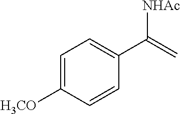 | 99 | |
| 30 | 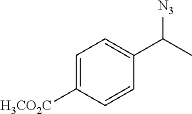 | 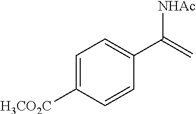 [SMILES: C=C(NC(C)=O)c1ccc(C(=O)OC)cc1] | 99 | |
| 31 | 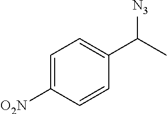 | 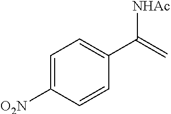 | 75 | |
| 32 | 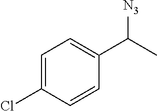 | 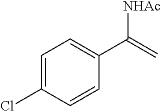 | 94 | |
| 33 | 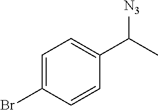 | 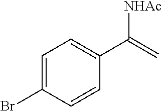 | 85 | |
| 34 | 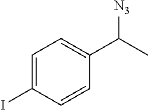 | 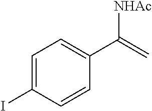 | 84 | |
| 35 | 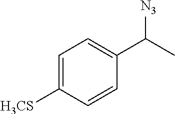 | 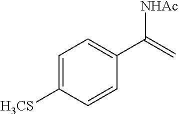 | 71 | |
| 36 | 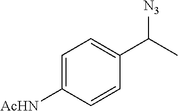 | 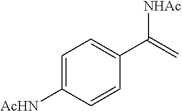 [SMILES: C=C(NC(C)=O)c1ccc(NC(C)=O)cc1] | 40 | |
| 37 | 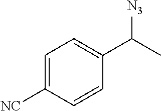 | 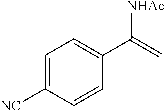 | 82 | |
| 38 | 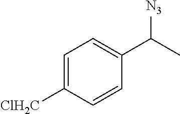 | 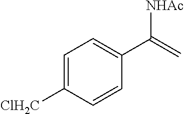 | 99 | |
| 39 | 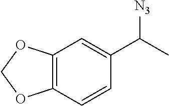 | 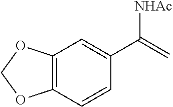 | 90 | |
| 40 | 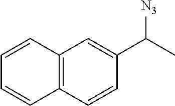 | 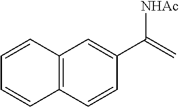 [SMILES: C=C(NC(C)=O)c1ccc2ccccc2c1] | 99 | |
| 41 | 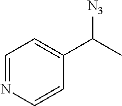 | 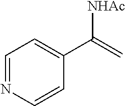 | 15 | |
| 42 | 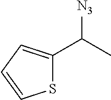 | 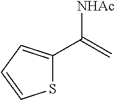 | 84 | |
| 43 | 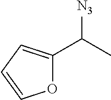 | 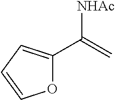 | 79 | |
| 44 | 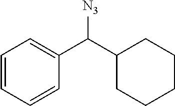 [SMILES: [N-]=[N+]=NC(c1ccccc1)C1CCCCC1] | 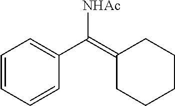 [SMILES: CC(=O)NC(=C1CCCCC1)c1ccccc1] | 99 | |
| 45 | 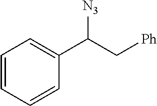 [SMILES: [N-]=[N+]=NC(Cc1ccccc1)c1ccccc1] | 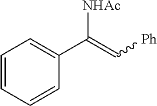 [SMILES: CC(=O)NC(=Cc1ccccc1)c1ccccc1] | 97 | 26:74 |
| 46 | 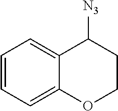 [SMILES: [N-]=[N+]=NC1CCOc2ccccc21] | 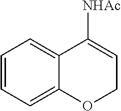 [SMILES: CC(=O)NC1=CCOc2ccccc21] | 85 | |
| 47 | 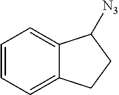 [SMILES: [N-]=[N+]=NC1CCc2ccccc21] | 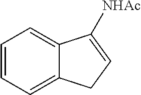 [SMILES: CC(=O)NC1=CCc2ccccc21] | 97 | |
| 48 | 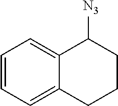 [SMILES: [N-]=[N+]=NC1CCCc2ccccc21] | 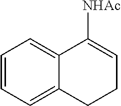 [SMILES: CC(=O)NC1=CCCc2ccccc21] | 95 | |
| 49 | 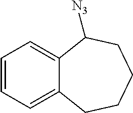 [SMILES: [N-]=[N+]=NC1CCCCc2ccccc21] | 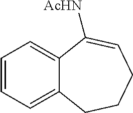 [SMILES: CC(=O)NC1=CCCCc2ccccc21] | 60 | |
| 50 | 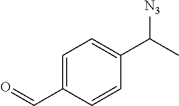 | 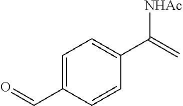 | 51 | |
| 51 | 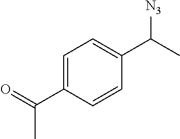 | 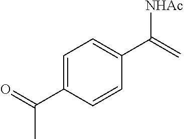 | 99 | |

The present invention relates to a method for preparing an enamide compound, which includes reacting an organic azide compound having α-hydrogen and an anhydride, and the reaction is able to be performed in a one-step process by addition of a specific ruthenium complex catalyst in the presence of an ionic liquid, has high reaction efficiency and an extended substrate spectrum, and provides a simple, practical, safe and environment-friendly synthesis method. Particularly, unlike the conventional methods, the present method does not need severe reaction conditions and a specific additive.

The enamide compound prepared by the reaction may be used as a useful synthesis starting material in various fields of medicine, materials, biochemistry and the like.

It would be understood by those of ordinary skill in the art that the above descriptions of the present invention are exemplary, and the exemplary embodiments disclosed herein can be easily modified into other specific forms without changing the technical spirit or essential features of the present invention. Therefore, it should be interpreted that the exemplary embodiments described above are exemplary in all aspects, and are not limitative.

What is claimed is:

1. A method for preparing an enamide compound, comprising:
reacting an organic azide compound having α-hydrogen represented by Formula 1 and an anhydride represented by Formula 2 by addition of a ruthenium complex catalyst in the presence of an ionic liquid, 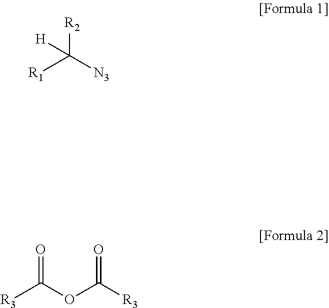
where R₁, R₂ and R₃ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group.

2. The method of claim 1, wherein the reaction is performed by addition of a ruthenium complex catalyst represented by Formula 3 or Formula 4: 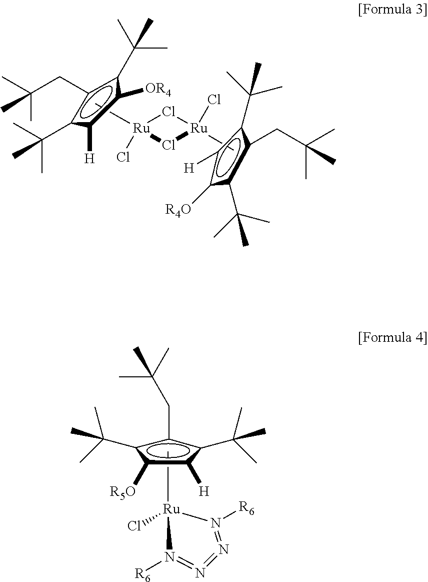
where R₄ is a substituted or unsubstituted C1 to C20 alkyl group, and R₅ is a substituted or unsubstituted C1 to C20 alkyl group, R₆ is a substituted or unsubstituted C1 to C20 alkyl group; a substituted or unsubstituted C6 to C20 aryl group; or a substituted or unsubstituted C3 to C20 heteroaryl group.

3. The method of claim 1, wherein the ruthenium complex catalyst is added at 1 to 5 mol % with respect to the organic azide compound having α-hydrogen.

4. The method of claim 2, wherein the ruthenium complex catalyst is added at 1 to 5 mol % with respect to the organic azide compound having α-hydrogen.

5. The method of claim 1, wherein the ruthenium complex catalyst and the anhydride are simultaneously added.

6. The method of claim 2, wherein the ruthenium complex catalyst and the anhydride are simultaneously added.

7. The method of claim 1, wherein the ruthenium complex catalyst is added, prior to addition of the anhydride, to produce an imine intermediate, and then the anhydride is added.

8. The method of claim 2, wherein the ruthenium complex catalyst is added, prior to addition of the anhydride, to produce an imine intermediate, and then the anhydride is added.

9. The method of claim 1, wherein the reaction is performed in the presence of an ionic liquid represented by Formula 5: 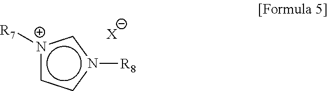
where R₇ and R₈ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group, and X is a halogen group.

10. The method of claim 1, wherein the reaction is performed at 50 to 120° C. for 30 minutes to 5 hours.

11. A ruthenium complex catalyst represented by Formula 3, which is used in the method for preparing an enamide compound of claim 1: 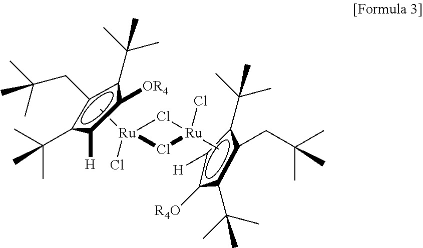
where R₄ is a substituted or unsubstituted C1 to C20 alkyl group.

12. The catalyst of claim 11, which is prepared by a reaction between a compound represented by Formula 6 and RuCl₃(H₂O)₃:

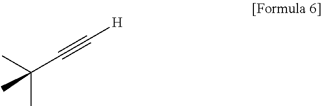

13. A ruthenium complex catalyst represented by Formula 4, which is used in the method for preparing an enamide compound of claim 1: 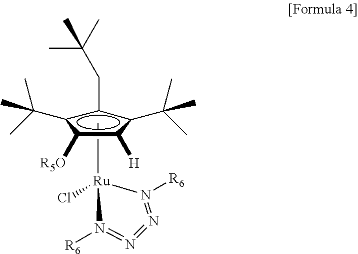
where R₅ is a substituted or unsubstituted C1 to C20 alkyl group, and R₆ is a substituted or unsubstituted C1 to C20 alkyl group; a substituted or unsubstituted C6 to C20 aryl group; or a substituted or unsubstituted C3 to C20 heteroaryl group.

14. The catalyst of claim 13, which is prepared by a reaction between a compound represented by Formula 3 and a compound represented by Formula 1: 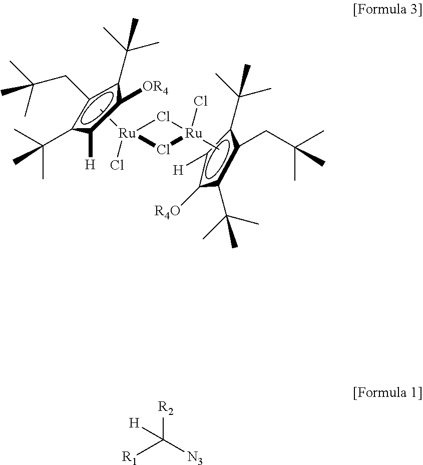
where R₄ is a substituted or unsubstituted C1 to C20 alkyl group, and R₁ and R₂ are the same or different from each other, each independently hydrogen; a substituted or unsubstituted C1 to C20 alkyl group; a substituted or unsubstituted C6 to C20 aryl group; or a substituted or unsubstituted C3 to C20 heteroaryl group.
